(19) United States
(12) Patent Application Publication
(10) Pub. No.: US 2025/0109081 A1
(43) Pub. Date: Apr. 3, 2025

(54) METHOD FOR OPERATING A MULTI-COMPONENT SYSTEM FOR PRODUCING HYDROCARBON PRODUCTS

(71) Applicant: Siemens Energy Global GmbH & Co. KG, München (DE)

(72) Inventors: Markus Kinzl, Dietzenbach (DE); Rüdiger Schneider, Eppstein (DE)

(21) Appl. No.: 18/729,194

(22) PCT Filed: Nov. 28, 2022
(86) PCT No.: PCT/EP2022/083395

§ 371 Date: Jul. 16, 2024

(30) Foreign Application Priority Data
Jan. 20, 2022 (DE) 10 2022 200 628.3

(51) Int. Cl. C07C 1/10; B01J 8/04

(52) U.S. Cl. CPC C07C 1/10; B01J 8/0492

(57) ABSTRACT

The invention relates to a method for operating a multi-component system for producing hydrocarbon products by means of regeneratively produced energy, wherein an inert gas is introduced into at least a first component of the system and wherein a process-internal gas is used as the inert gas.

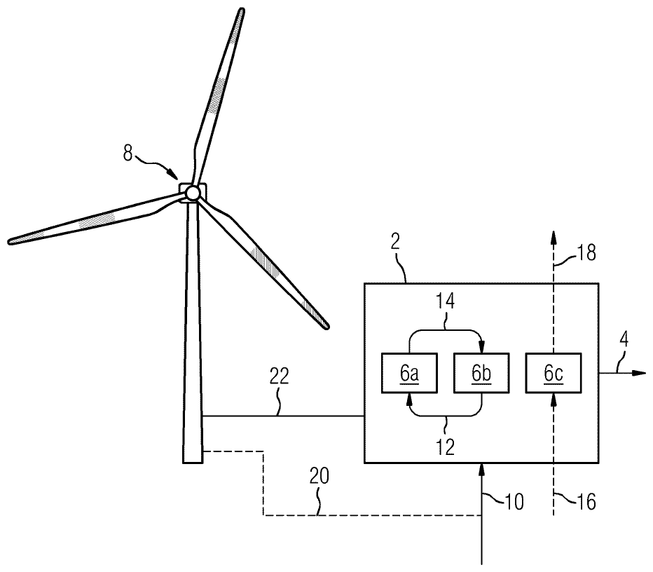

### BACKGROUND

The invention relates to a method of operating a multicomponent plant for production of hydrocarbon products by means of renewable energy.

The invention further relates to a multicomponent plant for production of hydrocarbon products by means of renewable energy.

Hydrocarbon products, for example fuels such as gasoline, diesel, kerosene or LPG, and likewise chemicals such as olefins, polymers etc., are produced by the use of renewable power (for example from wind or solar energy). First of all, hydrogen is produced, typically by the electrolysis of water, with occurrence of oxygen as by-product. The hydrogen is then reacted with CO₂, CO or CO₂/CO mixtures to give hydrocarbons, for example via the intermediate of methanol and further reaction thereof to give gasoline (methanol-to-gasoline, MtG), kerosene (methanol-to-kerosene, MtK), olefins (methanol-to-olefins, MtO) or other hydrocarbon products.

An alternative known synthesis route is Fischer-Tropsch synthesis, which at first affords a synthetic oil (“syncrude”), which can be converted by refinery processes to the hydrocarbon products mentioned or others.

Alternatively, hydrogen and CO/CO₂ can be converted by fermentation to ethanol, the dehydration of which forms ethylene, which can be converted by oligomerization, polymerization, copolymerization and a wide variety of different chemical reactions to a multitude of chemical/petrochemical products, for example to fuels such as gasoline or kerosene.

These and other power-to-X (PtX) processes in which renewable energy is used to produce synthetic hydrocarbons are all addressed by the present invention.

In processes for producing hydrocarbons, process-extrinsic inert gases are commonly introduced at various points in order to inertize vessels or other apparatuses. An important special case is the regeneration of catalysts, for example by burnoff of coke by feeding in air or oxygen, wherein the burnoff has to be preceded by removal of the hydrocarbons present in the relevant apparatus by purging with an inert gas in order to avoid the formation of explosive mixtures with air or oxygen.

“Inert gases” refer here to gases that are not involved in the reactions that occur in the PtX process, but serve in particular as carrier/purge/inertization gases. What is meant here by “process-extrinsic” is that they are not present in the plant because of the PtX reactions on the reactant side or product side or for any other reason, but are introduced additionally.

Process-extrinsic inert gases are introduced intentionally or unintentionally into the process: intentionally normally for inertization of vessels or plant components; unintentionally as dissolved gases in the reactants fed to the process. The process-extrinsic inert gases are ultimately discharged again from the process without involvement in any reactions, for example as constituents of products or by-products, frequently also as a constituent of an offgas which is discharged as purge stream (and is possibly sent to utilization outside the PtX process).

The process-extrinsic inert gases dissolve partly in the liquid hydrocarbon products (intermediates or end products) and mix with the hydrocarbon gas phase which either itself goes into a (by-) product or into the offgas.

The introduction of process-extrinsic inert gases is associated with serious drawbacks. The products (e.g. gasoline, kerosene or hydrocarbon chemicals) are laden with the process-extrinsic inert gases, which are unwanted with regard to product quality. Depending on the demands on product quality, it may be necessary to remove them by separation processes before the sale of the products. Particularly the presence of nitrogen, which is the most commonly used process-extrinsic inert gas for reasons of cost, leads to a further significant drawback, since nitrogen, which is virtually always gaseous in the relevant processes because of its low boiling point, accumulates in the offgas and can be very troublesome in the course of further utilization thereof. The offgas, which is very high in energy because of the high concentrations of hydrocarbons present, is typically to be utilized for energy purposes by combustion. In the case of PtX processes, one option is combustion with the oxygen obtained as by-product by electrolysis, which may in principle be associated with two benefits. Firstly, by contrast with combustion with air, there is no formation of NOx compounds, which are subject to strict emissions limits (reduction of the level of any NOx compounds would necessitate a costly deNOx plant component). Secondly, the CO and CO₂ products formed by the hydrocarbon offgas combustion can be fed back to the PtX synthesis process as reactants. It should be taken into account here that the provision of renewable CO/CO₂ accounts for a significant proportion of the costs of PtX processes.

However, the benefits mentioned are negated when nitrogen is present in the hydrocarbon offgas since, firstly, offgas incineration gives rise to NOx compounds and, secondly, these NOx compounds and the nitrogen itself, together with the CO/CO₂ mixtures that are reusable as reactants, get into the PtX process, where they would accumulate without the discharge of relatively high proportions of the recycling stream. This is associated with a serious drawback, since any discharge should be reduced to a minimum because discharges are always associated with loss of CO/CO₂, the main components of offgas utilization.

The alternatively possible use of argon or another inert gas does prevent the formation of NOx compounds, but these inert gases also get into the CO/CO₂ product, and hence the described discharge of significant gas volumes (including CO/CO₂) is necessary. Moreover, argon and other process-extrinsic inert gases are much more costly than nitrogen, which can be produced comparatively inexpensively by air fractionation, and so the use thereof is not normally an option in any case for economic reasons.

In summary, it can be stated that the use of process-extrinsic inert gases in PtX processes for production of renewable hydrocarbons is associated with distinct drawbacks. The inertization of vessels and other apparatuses or plant components is generally indispensable in the synthesis of hydrocarbons.

A prominent example is the inertization of vessels having fill levels that fluctuate over time (greater or lesser fluctuations in fill level occur in virtually all vessels). If the liquid level in a vessel falls, the vessel pressure is maintained by adding a gas that must be compatible with the substances present in the vessel (no chemical reactivity, minimum solubility etc.).

The inertization of plant components is also a typical step in the startup of a plant, since the vessels and apparatuses are usually filled with air after the assembly of the plant.

A further important example is the regeneration of catalysts. Owing to the typically high temperatures (>200° C., often >300° C.) in the region of the catalyst surfaces where the strongly exothermic reaction steps of the processes for hydrocarbon synthesis take place, side reactions occur, including decomposition of carbonaceous components and formation of a coke layer on the catalyst surface, which leads to increasing deactivation. For regular regeneration of the catalyst, the coke layer is burnt off with air or oxygen from time to time (typically after a few days, depending on the process). Before addition of the (atmospheric) oxygen, the hydrocarbons present in the reactor are removed by purging with an inert gas in order to avoid the formation of explosive mixtures.

US2013137783 A1 discloses a system for recycling of industrial CO₂ emissions for non-intermittent production of renewable fuels and chemicals via gaseous intermediates that utilize intermittent renewable energies and renewable carbonaceous raw material. In this system, a concentrated CO/CO₂ recycling stream is used to adjust a synthesis gas mixture.

### SUMMARY

It is an object of the invention to propose an alternative method of intentional introduction of gases not involved in the PtX process itself in a PtX process for production of renewable hydrocarbons with regard to inertization of plant components and/or regeneration of catalysts.

The object is achieved in accordance with the invention by a method of operating a multicomponent plant for production of hydrocarbon products by means of renewable energy, wherein an inert gas from a second component of the plant is intentionally introduced at least into a first component of the plant for inertization of plant components and/or for regeneration of catalysts, wherein the inert gas used is a process-intrinsic gas and wherein a first inert gas offgas stream is recycled from the first component of the plant into the process for producing hydrocarbon products.

The object is additionally achieved by a multicomponent plant for production of hydrocarbon products by means of renewable energy, with a first conduit for introduction of an inert gas for inertization of plant components and/or for regeneration of catalysts into a first component of the plant from a second component of the plant, wherein the inert gas used is a process-intrinsic gas, and with an intermediate conduit for recycling of a first inert gas offgas stream from the first component into the process for producing hydrocarbon products.

### DETAILED DESCRIPTION

The benefits and preferred configurations cited hereinafter in relation to the method can be applied mutatis mutandis to the plant.

What is meant here by “process-intrinsic” is a gas which is already present in the plant and which is now additionally being used as carrier/purge/inertization gas. It is explicitly pointed out that the invention relates not just to the addition of the abovementioned gases in the course of inertization processes, but to all possible intentional introductions of gases within PtX processes, for whatever reason these introductions are implemented.

“Components of the plant” means parts of the plant that are functionally or spatially separated from one another. A component may, for example, be a reactor, a vessel, a heat exchanger or a separator.

The term “catalysts” encompasses not only the multitude of catalysts, but may likewise refer to a single catalyst.

According to the invention, inertizations, especially of vessels or prior to the regeneration of catalysts, are no longer conducted with process-extrinsic inert gases, but rather with process-intrinsic gases. The recycling of the process-intrinsic gases here does not serve for reuse of these as reactants in the synthesis of the hydrocarbon products; instead, they are merely used in association with the inertization of plant components and/or regeneration of one or more catalysts. It can be assumed that portions of the gases used for the inertization will get into the product and/or into the process offgas. They must not cause any harmful effects there, for example in the firing of offgas with electrolysis oxygen, the intention of which, apart from the recovery of thermal energy, is also the generation of CO/CO₂ mixtures that are returned to the process as reactants. The inert gases used or combustion products thereof ideally occur in the process in any case, and so the addition thereof does not introduce any new process-extrinsic components.

The inert gases used additionally have the following benefits:

High compatibility with the substances present in the relevant apparatuses, which also include catalysts.

They are inexpensive gases, the use of which does not significantly reduce the economic viability of the process.

According to the invention, a first inert gas offgas stream from the first component of the plant is recycled into the process for producing hydrocarbon products. The recycling is especially accomplished after firing with oxygen or another treatment. Because of the use of process-intrinsic inert gases, the recycling of the inert gases that are still present after the offgas firing or combustion products thereof together with CO/CO₂ into the process is not associated with any unwanted accumulation.

In one embodiment, the process-intrinsic gas used is carbon dioxide, CO₂. CO₂ occurs in any case at various points in the relevant PtX processes; the accumulation thereof in the offgas and in the combustion products thereof that can be returned to the process as reactants is in no way disadvantageous. CO₂ is usable particularly advantageously for several reasons:

CO₂ is chemically relatively inert, and so there is no chemical reaction in the inertization of apparatuses, for example with hydrocarbon liquids being stored in the vessels to be inertized, or over or with the catalysts to be regenerated.

CO₂ as a constituent of the offgas does not react in the firing; the presence of CO₂ as diluent gas can even be advantageous for the firing process because the combustion temperature can be reduced by diluting inert gases.

CO₂ is available in any case as a reactant in PtX plants, usually in a storage tank from which it is drawn. It is therefore not specially transported in; the costs for CO₂ are comparatively low for that reason among others.

CO₂ may be stored in liquid or gaseous form. When used for inertization, it is used in the gaseous state. Under typical use conditions, CO₂ is gaseous.

In another embodiment, the process-intrinsic inert gas used is water vapor. The introduction of water vapor has benefits but also limits by comparison with CO₂, and so use is possible or advisable in particular cases:
Water is chemically relatively inert, and so there is no chemical reaction in the inertization of apparatuses, for example with hydrocarbon liquids being stored in the vessels to be inertized. However, it is disadvantageous to use water vapor where water condenses because of the given temperature/pressure conditions (condensed water is undesirable, for example, in hydrocarbon storage tanks, where a second liquid phase would form). Moreover, various catalysts, for example the catalyst used in the MtG process (typically zeolite catalysts, e.g. ZSM-5), are water-sensitive to some degree, and so water vapor should not be used here. Thus, water vapor is an option only in particular cases. In general, water vapor can be used when no steam-sensitive components are present, for example in separators, in which there is a water phase in any case in normal operation.

Water vapor as a constituent of the offgas does not react on firing; the presence of water vapor as diluent gas may even be advantageous because the combustion temperature can be reduced by diluting inert gases. The recycling of water as a constituent of the CO/CO₂ mixture as reactants into the PtX process has the disadvantage that water is not a reactant but a product of the hydrocarbon syntheses, and so the presence thereof adversely affects the reaction equilibrium. For that reason, separation of the water from the CO/CO₂ mixture is required, which is achievable effectively, for example, in the form of condensation of the water.

One advantage of water is that—disregarding desert regions—it is usually available in large volumes and no separate transport is required. The demineralization of water is effected in any case in the region of PtX plants since deionized water constitutes the starting material for hydrogen recovery by electrolysis.

It is advisable to store water in liquid form. When used for inertization, it first has to be evaporated.

In a further embodiment, the process-intrinsic inert gas used is carbon monoxide, CO. The introduction of CO is possible or advisable in particular cases too. CO is more chemically reactive overall than CO₂ and water, but no reactions with hydrocarbon liquids present in the components to be inertized are to be expected. In principle, therefore, CO can be used for the inertization of these apparatuses. In this case, it should be ensured that, when the liquid fill level rises, the displaced, toxic CO flowing out does not get into the atmosphere. It is rendered harmless as part of the offgas, for example by combustion to CO₂. Reactions of CO with catalysts can occur to some degree (for example formation of metal carbonyls, although the formation is reversible). The catalyst used in the MtG process is an example of a catalyst that does not react with CO; thus, CO would especially be suitable for inertization in this process. CO will preferably be used when no CO-sensitive catalyst or no catalyst at all is present, i.e., for example, in all separation apparatuses such as separators, separating columns or storage vessels.

A further aspect that supports the use of CO is that CO, as a possible constituent of the offgas, on firing thereof, is oxidized to CO₂ and hence can be recycled into the PtX process as a reactant.

CO is stored intermediately as reactant in some PtX processes and is available in large volumes in these cases (for example for Fischer-Tropsch applications). No separate transport of CO is required; CO is thus a relatively inexpensive inertizing agent. In these cases, it is stored in gaseous form and can be used without further pretreatment.

In exemplary embodiments, a process-extrinsic inert gas, for example nitrogen, argon or another inert gas, is introduced into a further component of the plant. In this case, there is a combination of process-intrinsic with process-extrinsic gases that are used at different points in the plant. In particular processes, nitrogen, argon or other process-extrinsic inert gases are indispensable in some parts of the plant as inertizing agent or for other reasons, for example if all gases present in any case in the process, for example CO₂, water vapor or CO, are unsuitable for purging of a catalyst-filled reactor. In that case, however, the remaining portion of the plant is inertized with one of the mentioned gases that occur in the process.

As mentioned, process-extrinsic inert gases can also get into the process unintentionally, for example dissolved in one of the reactants. By way of illustration, reference is made to the possible use of biomass as CO₂ source, as a result of which certain sulfur and N₂ impurities get into the PtX process together with the CO₂. In order to prevent accumulation of these components above an acceptable degree, a particular amount of the offgas is discharged continuously or semi-continuously from the PtX process as what is called a purge stream. The discharged components are lost from the process. However, the process-extrinsic inert gases that are unintentionally introduced into the process are typically present in very small concentrations; for example, the CO₂ obtained from biomass is pre-purified before use in the PtX process in order to remove sulfur in particular. The inert gas concentrations present after unintentional introduction of inert gas into the PtX process are therefore several times smaller than in the processes used to date, in which the gases are added deliberately, and so the purge stream is also correspondingly much smaller than in prior art processes.

In exemplary embodiments, a second inert gas offgas stream is generated in the further component of the plant, which is conducted out of the plant, especially separately from the first inert gas offgas stream. The offgas from the part of the process in which the process-extrinsic inert gas is used is discharged separately from the PtX process, such that the inert gases cannot accumulate in the process. Only the offgases from the parts of the plant free of the process-extrinsic inert gases, especially after firing with oxygen or another treatment, are recycled into the PtX process, such that no accumulation of process-extrinsic inert gases can occur. In this process variant too, furthermore, a small purge stream is possible if process-extrinsic inert gases unintentionally get into the PtX process. Here too, this purge stream, for the reasons mentioned, will be much smaller than in processes known to date.

The invention is elucidated in detail by way of example with reference to a drawing. The sole figure herein shows a schematic of a multicomponent power-to-X plant 2 for production of hydrocarbon products 4, for example methanol, gasoline, kerosene etc. Individual components of the plant are identified by reference numerals 6a, 6b, 6c. For production of the hydrocarbon products 4, energy from a renewable energy source is used, for example from a wind turbine 8, a photovoltaic system etc., with use of the energy for production of reactants 10 for the PtX process (indicated by the dotted line 20) and/or directly for operation of the plant 2 (shown by line 22).

In a first component 6a of the plant 2, an inert gas 12 is required, which is not used directly as reactant but has a different function, for example as fill gas or purge gas. In this case, a process-intrinsic gas, especially CO₂, CO or water vapor, is taken from a second component 6b of the plant 2 and introduced into the first component 6a via a first conduit. Finally, a first inert gas offgas stream 14 is returned to the process for producing hydrocarbon products via an intermediate conduit from the first component 6a, which is indicated schematically by the arrow 14. This inert gas offgas stream 14 is optionally treated before being returned to the process: for example by combustion with electrolysis O₂, forming CO and/or CO₂, which are in turn returned to the process.

Under some circumstances, a process-extrinsic inert gas, for example nitrogen, argon or another inert gas, can be introduced into a further component 6c of the plant 2, if the use of a process-intrinsic inert gas is not possible or optimal. This is shown by the dotted arrow 16. A further inert gas offgas stream 18 is generated here in the further component 6c, which is discharged separately from the plant 2 without mixing of the first inert gas offgas stream 14 and the further inert gas offgas stream 18.

In summary, in the procedure of the invention, inert gases having the abovementioned properties such as compatibility with the substances present in the relevant apparatuses/the gases added are not process-extrinsic/the gases added are inexpensive are used. A particularly advantageously usable gas is CO₂; water vapor and/or CO are also options in some cases. Ideally, these gases are used at all points in the PtX process where addition of inert gas is necessary, typically in the context of inertization steps. If the addition of process-extrinsic gases should be necessary at individual points, the process-extrinsic gases are then added only at exactly those points, whereas process-intrinsic gases are introduced at all other points.

The proposed method has a number of advantages:

The products are not contaminated by process-extrinsic gases, and subsequent purification of the products for removal of process-extrinsic gases can be dispensed with.

There are no process-extrinsic inert gases in the offgas from the PtX plant, and hence not in the offgas combustion products either that are formed on firing with oxygen.

No formation of NOx compounds formed when N₂ is used according to the prior art.

In the offgas combustion product, there are no inert gases that are recycled into the PtX process together with CO₂/CO. Thus, it is possible to avoid a massive discharge (purge) of recycling gas that would be necessary if any great amounts of inert gas were to accumulate in the offgas and hence also in the process. Prevention or minimization of discharge likewise avoids, or at least keeps to a minimum, the inevitably accompanying loss of valuable hydrocarbonaceous components.

If a purge stream is needed to remove small amounts of inert gas from the PtX process, for example in the case of unintentional inert gas input, this purge stream can be kept very small, and so the loss of valuable C-containing components associated with a purge can be minimized.

If no process-extrinsic inert gas is added, it is unnecessary to transport such a gas to the PtX plant, and no additional infrastructure has to be provided to deal with this gas (e.g. gas storage, gas metering, possibly air fractionation for nitrogen production, etc.), which enables cost savings.

In the firing of PtX offgases, the presence of an inert gas such as CO₂ or water vapor can have an advantageous effect, since diluting inert gases lower the combustion temperature, which reduces thermal stress on the burner.

1. A method of operating a multicomponent plant for production of hydrocarbon products by means of renewable energy, wherein an inert gas from a second component of the multicomponent plant is intentionally introduced at least into a first component of the multicomponent plant for inertization of plant components and/or for regeneration of catalysts, wherein the inert gas used is a process-intrinsic inert gas and wherein a first inert gas offgas stream is recycled from the first component of the multicomponent plant into a process for producing hydrocarbon products.

2. The method as claimed in claim 1, wherein the process-intrinsic inert gas used is carbon dioxide CO₂.

3. The method as claimed in claim 1, wherein the process-intrinsic inert gas used is water vapor.

4. The method as claimed in claim 1, wherein the process-intrinsic inert gas used is carbon monoxide CO.

5. The method as claimed in claim 1, wherein a process-extrinsic inert gas, for example nitrogen, argon or another inert gas, is introduced into a further component of the multicomponent plant.

6. The method as claimed in claim 5, wherein a further inert gas offgas stream is generated in the further component of the multicomponent plant and is led out of the multicomponent plant.

7. A multicomponent plant for production of hydrocarbon products by means of renewable energy, with a first conduit for introduction of an inert gas for inertization of plant components and/or for regeneration of catalysts into a first component of the multicomponent plant from a second component of the multicomponent plant, wherein the inert gas used is a process-intrinsic gas, and with an intermediate conduit for recycling of a first inert gas offgas stream from the first component into a process for producing hydrocarbon products.
